(19) United States
(12) Patent Application Publication
(10) Pub. No.: US 2025/0136933 A1
(43) Pub. Date: May 1, 2025

(54) COMPOSITIONS AND METHODS FOR PRODUCING PRIMORDIAL GERM CELL-LIKE CELLS

(71) Applicants: The Regents of the University of California, Oakland, CA (US); GlaxoSmithKline Intellectual Property Development Limited, Stevenage (GB)

(72) Inventors: Kaivalya Shevade, Oakland, CA (US); Rustam Esanov, San Francisco, CA (US); Yeqing Angela Yang, San Francisco, CA (US); Volkan Sevim, San Francisco, CA (US); Laralynne Przybyla, Oakland, CA (US); Shawn Shafer, San Francisco, CA (US)

(21) Appl. No.: 18/930,210

(22) Filed: Oct. 29, 2024

Related U.S. Application Data
(60) Provisional application No. 63/546,733, filed on Oct. 31, 2023.

(51) Int. Cl. C12N 5/0735; C12N 9/22; C12N 15/11; C12N 15/90

(52) U.S. Cl. CPC C12N 5/0611; C12N 9/22; C12N 15/11; C12N 15/907; C12N 2310/20; C12N 2506/45

(57) ABSTRACT

Methods and compositions are provided for producing a primordial germ cell like cell (PGCLC) from a pluripotent stem cell, e.g., an induced pluripotent stem cell (iPSC), via GATA5 overexpression. GATA5 expression can be induced using any convenient method, e.g., by introducing an exogenous nucleic acid encoding GATA5 or by using a tool such as CRISPRa to stimulate expression from the GATA5 endogenous locus.

### CROSS-REFERENCE

This application claims the benefit of U.S. Provisional Patent Application No. 63/546,733 filed Oct. 31, 2023, which application is incorporated herein by reference in its entirety.

### INCORPORATION BY REFERENCE OF SEQUENCE LISTING PROVIDED AS AN XML FILE

A Sequence Listing is provided herewith as a Sequence Listing XML, “UCSF-758WO_SEQ_LIST.xml” created on Oct. 28, 2024 and having a size of 4, 131 bytes. The contents of the Sequence Listing XML are incorporated by reference herein in their entirety.

### I. INTRODUCTION

Primordial Germ Cells (PGCs) are the early embryonic precursors of the germ cell lineage which can further differentiate into mature oocytes or sperm in females and males respectively. Thus, PGCs are common ancestors of all germline cells. In mammals, PGCs emerge in early-stage embryos around the timing of gastrulation at or near epiblast, and specification of PGCs from their precursor cells involves multiple growth factors secreted by adjacent cells. PGCs undergo extensive epigenetic modifications including global depletion of DNA methylation while differentiation to healthy gametes to ensure that appropriate epigenetic information is transmitted across generations.

Understanding the mechanisms of germ cell lineage development and differentiation is important in reproductive medicine for the development of therapies against fertility problems in humans. PGCs are a transitionary cell type during development making it difficult if not impossible to obtain primary human PGCs. Advancements in germline stem cell biology have made it possible to generate PGC-like cells from human and mouse pluripotent stem cells. For example, protocols have been recently developed to differentiate induced pluripotent stem cells (iPSCs) into Primordial Germ Cell Like Cells (PGCLCs), e.g., by mimicking the embryonic growth factor environment in vitro.

More efficient compositions and methods are needed for producing primordial germ cells, and such are provided herein.

### II. SUMMARY

The inventors have made the surprising finding that Primordial Germ Cell like Cells (PGCLCs) can be generated from pluripotent stem cells (PSCs), e.g., from induced pluripotent stem cells (iPSCs), by overexpressing the transcription factor GATA binding protein 5 (GATA5). This approach is advantageous compared to existing methods due to its speed, scalability, efficiency and low batch-to-batch variability. The compositions and methods provided herein will allow rapid generation of PGCLCs from PSCs.

Provided are compositions and methods for producing a PGCLC from a PSC (e.g., from an iPSC). A subject method includes inducing GATA5 expression (i.e., overexpressing GATA5) in a PSC. In some cases, GATA5 expression is induced (i.e., GATA5 is overexpressed) in an iPSC.

In some embodiments, overexpressing GATA5 (i.e., stimulating/inducing GATA5 expression) includes expressing, in a PSC (e.g., an iPSC), a CRISPRa fusion protein that, in the presence of a guide RNA, stimulates expression of GATA5—in some cases GATA5 expression is induced from the GATA5 endogenous locus. In some such cases, a nucleotide sequence encoding the CRISPRa fusion protein is operably linked to an inducible promoter, and a subject method includes stimulating expression of the CRISPRa fusion protein from the inducible promoter. In some cases, the nucleotide sequence encoding the CRISPRa fusion protein is integrated into the genome of the PSC. In some cases, the CRISPRa fusion protein includes dCas9 fused to VPR. In some cases, a subject method includes introducing the guide RNA or a nucleic acid encoding the guide RNA into the PSC.

In some embodiments, overexpressing GATA5 (i.e., stimulating/inducing GATA5 expression) includes introducing a nucleic acid (e.g., an exogenous nucleic acid such as a recombinant expression vector) encoding GATA5 into the PSC (e.g., an iPSC). For example, overexpressing GATA5 can include contacting the PSC with a virus that delivers the nucleic acid encoding GATA5 to the PSC. In some cases, the nucleic acid encoding GATA5 is an RNA—and as such the method includes introducing an RNA encoding GATA5 into the PSC.

In some embodiments, a subject method further includes measuring expression of one or more PGC markers, after GATA5 overexpression, to determine whether a PGCLC has been produced. Examples of PGC markers include, but are not limited to: EOMES, SOX17, PRDM1, TFAP2C, GATA3, and NANOS3, and any combination thereof. In some case, the PGC markers used include EOMES, SOX17, PRDM1, TFAP2C, GATA3, or any combination thereof.

In some cases, the PSC (e.g., an iPSC) is a human cell, and in some cases the PSC (e.g., an iPSC) was derived from a human patient. It is to be understood that whenever an embodiment(s) is disclosed herein in the context of a PSC, that same embodiment(s) is also contemplated/disclosed for an iPSC, and vice versa.

Reagents, compositions, and kits/systems that find use in practicing the subject methods are provided.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A-1C GATA5 overexpression in iPSCs led to expression of Primordial Germ Cell markers. FIG. 1A) Single cell multiomic analysis shows that GATA5 overexpressing cells express PGC markers EOMES, SOX17, PRDM1 and TFAP2C. FIG. 1B) Volcano plot from bulk RNA-seq analysis listing differentially expressed genes between GATA5 overexpressing cells and control iPSCs. FIG. 1C) Bar-graph from bulk RNA-seq experiments showing upregulation of PGC markers in GATA5 overexpressing cells and compared to iPSCs.

### IV. DEFINITIONS

A DNA sequence that “encodes” a particular RNA is a DNA nucleic acid sequence that is transcribed into RNA. A DNA polynucleotide may encode an RNA (mRNA) that is translated into protein, and the DNA can therefore be said to encode the protein. A DNA may encode an non-coding RNA (ncRNA), i.e., and RNA that is not translated into protein (e.g. tRNA, rRNA, CRISPR/Cas guide RNA).

A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. Eukaryotic promoters will often, but not always, contain “TATA” boxes and “CAT” boxes. Various promoters, including inducible promoters, may be used to drive the various vectors of the present invention. The promoter may be a constitutively active promoter, i.e. a promoter is active in the absence externally applied agents (e.g., CMV, EF1a, beta-Actin), or it may be an inducible promoter (e.g., T7 RNA polymerase promoter, heat shock promoter, Tetracycline-regulated promoter, Steroid-regulated promoter, Metal-regulated promoter, doxycycline-regulated promoter, etc). As used herein, an inducible promoter is a promoter whose activity is regulated by a factor that induces expression, e.g., upon the application of an agent to the cell, (e.g. doxycycline), the induced presence of a particular RNA polymerase (e.g., T7 RNA polymerase), and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., a guide RNA) or a coding sequence (e.g., GATA5, a CRISPRa fusion protein) and/or regulate translation of an encoded polypeptide.

“Exogenous,” is used herein to refer to something not endogenous to the cell. For example, when an expression vector encoding GATA5 and/or a CRISPRa fusion protein is delivered to a cell, the expression vector is exogenous to the cell—the expression vector is an exogenous nucleic acid.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms. Alternatively, DNA sequences encoding RNA (e.g., a guide RNA) that is not translated may also be considered recombinant. Thus, e.g., the term “recombinant” polynucleotide or “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

A “vector” or “expression vector” is a replicon, such as plasmid, phage, virus, or cosmid, to which another DNA segment, i.e. an “insert”, may be attached so as to bring about the replication and/or expression of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression. The coding sequence can also be said to be operably linked to the promoter.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

A cell has been “genetically modified” or “transformed” or “transfected” by exogenous DNA, e.g. a recombinant expression vector, when such DNA has been introduced inside the cell. The presence of the exogenous DNA results in permanent or transient genetic change. The transforming DNA may or may not be integrated (covalently linked) into the genome of the cell. A stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

Suitable methods of genetic modification (also referred to as “transformation”) include viral infection (transduction), transfection, electroporation, particle gun technology, calcium phosphate precipitation, direct microinjection, and the like. The choice of method is generally dependent on the type of cell being transformed and the circumstances under which the transformation is taking place (e.g., in vitro, ex vivo, or in vivo). A general discussion of these methods can be found in Ausubel, et al., Short Protocols in Molecular Biology, 3rd ed., Wiley & Sons, 1995.

### V. DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Compositions and Methods

As noted above, provided are compositions and methods for producing a primordial germ cell like cell (PGCLC). Such methods include inducing GATA5 expression (i.e., overexpressing GATA5) in a pluripotent stem cell (PSC), such as in an induced pluripotent stem cell (iPSC), thereby producing a PGCLC.

i. GATA5

GATA binding protein 5 (GATA5) is also sometimes known as CHTD5; GATAS; bB379024.1. Human GATA5 has the following amino acid sequence:

(NCBI No. NP_536721.1; Uniprot No. Q9BWX5)
(SEQ ID NO: 1)
MYQSLALAASPRQAAYADSGSFLHAPGAGSPMFVPPARVPSMLSYLSGC
EPSPQPPELAARPGWAQTATADSSAFGPGSPHPPAAHPPGATAFPFAHS
PSGPGSGGSAGGRDGSAYQGALLPREQFAAPLGRPVGTSYSATYPAYVS
PDVAQSWTAGPFDGSVLHGLPGRRPTFVSDFLEEFPGEGRECVNCGALS
TPLWRRDGTGHYLCNACGLYHKMNGVNRPLVRPQKRLSSSRRAGLCCTN
CHTTNTTLWRRNSEGEPVCNACGLYMKLHGVPRPLAMKKESIQTRKRKP
KTIAKARGSSGSTRNASASPSAVASTDSSAATSKAKPSLASPVCPGPSM
APQASGQEDDSLAPGHLEFKFEPEDFAFPSTAPSPQAGLRGALRQEAWC
ALALA

ii. Pluripotent Stem Cells (e.g., iPSCs)

The term “stem cell” is used herein to refer to a mammalian cell that has the ability both to self renew and to generate a differentiated cell type (see, e.g., Morrison et al. (1997) Cell 88:287-298). In the context of cell ontogeny, the adjective “differentiated”, or “differentiating” is a relative term. A “differentiated cell” is a cell that has progressed further down the developmental pathway than the cell it is being compared with. Thus, pluripotent stem cells (described below) can differentiate into further restricted stem cells (e.g., Epiblast stem cells (described below), mesodermal stem cells, mesenchymal stem cells, and the like), which in turn can differentiate into cells that are further restricted (e.g., cardiomyocyte progenitors, neural progenitors, and the like), which can differentiate into end-stage cells (i.e., terminally differentiated cells, e.g., neurons, skeletal muscle cells, cardiomyocytes, adipocytes, osteoblasts, and the like), which play a characteristic role in a certain tissue type, and may or may not retain the capacity to proliferate further. Different types of stem cells may be characterized by both the presence of specific markers (e.g., proteins, RNAs, etc.) and the absence of specific markers. Stem cells may also be identified by functional assays both in vitro and in vivo, particularly assays relating to the ability of stem cells to give rise to particular types of differentiated progeny.

The term “pluripotent stem cell” or “PSC” is used herein to mean a stem cell capable of self renewal and of producing all cell types of the organism (i.e., it is pluripotent). Therefore, a PSC can give rise to cells of all germ layers of the organism (e.g., the endoderm, mesoderm, and ectoderm). PSCs may be in the form of an established cell line, they may be obtained directly from primary embryonic tissue, or they may be derived from a somatic cell. Because the term PSC refers to pluripotent stem cells regardless of their derivation, the term PSC encompasses the terms embryonic stem cell (ESC), induced pluripotent stem cell (iPSC), embryonic germ stem cell (EGSC), and epiblast stem cells (EpiSC). A human PSC can be referred to as an “hPSC”, an “hESC”, an “hiPSC”, and the like, depending on the context and the derivation of the PSC. Likewise, a mouse PSC can be referred to as an “mPSC”, an “mESC”, an “miPSC”, an mEpiSC, and the like. The methods described herein are applicable to any mammalian PSC, including but not limited to an ESC, an iPSC, an EpiSC, and/or an EGSC.

The PSCs of interest are mammalian, where the term mammalian refers to a cell from any animal classified as a mammal, including humans, domestic and farm animals, and zoo, laboratory, sports, or pet animals, such as dogs, horses, cats, cows, mice, rats, rabbits, etc. In some embodiments, the mammal is a human and the mammalian PSC is therefore a human PSC. In some cases, the PSC was derived from a human patient.

By “induced pluripotent stem cell” or “iPSC” it is meant a PSC that is derived from a cell that is not a PSC (i.e., from a cell this is differentiated relative to a PSC). iPSCs can be derived from multiple different cell types, including progenitor cells as well as terminally differentiated cells, and methods of producing iPSCs will be known to one of ordinary skill in the art. (see, e.g., Takahashi et. al, Cell. 2007 Nov. 30; 131 (5): 861-72; Takahashi et. al, Nat Protoc. 2007; 2 (12): 3081-9; Yu et. al, Science. 2007 Dec. 21; 318 (5858): 1917-20. Epub 2007 Nov. 20). iPSCs have an ES cell-like morphology, growing as flat colonies with large nucleo-cytoplasmic ratios, defined borders and prominent nuclei. In addition, like other PSCs, iPSCs exhibit one or more markers of pluripotency known by one of ordinary skill in the art, including but not limited to Alkaline Phosphatase activity, SSEA3 expression, SSEA4 expression, Sox2 expression, Oct3/4 expression, Nanog expression, etc. Examples of methods of generating and characterizing iPSCs may be found in, for example, U.S. Patent Publication Nos. US20090047263, US20090068742, US20090191159, US20090227032, US20090246875, and US20090304646, the disclosures of which are incorporated herein by reference.

Generally, to generate iPSCs, somatic cells are provided with a cocktail (i.e., combination) of reprogramming factors (selected from, for example, Oct3/4, Sox2, Klf4, c-Myc, Nanog, Lin28, etc.) known in the art to reprogram the somatic cells to become pluripotent stem cells. By “reprogramming factors” it is meant one or more, i.e., a cocktail, of biologically active factors that act on a cell to alter transcription, thereby reprogramming a cell to pluripotency. For example, in some cases the ‘Yamanaka factors’ (also referred to as OSKM) (Oct3/4, Sox2, Klf4, c-Myc) are used. In some cases, Nanog and Lin28 are also used. In some cases, Oct4, Sox2, Nanog, and Lin28 are used. When reprogramming factors are provided to cells (i.e., cells are contacted with reprogramming factors), these reprogramming factors may be provided to the cells individually or as a single composition, that is, as a premixed composition, of reprogramming factors. The factors may be provided at the same molar ratio or at different molar ratios, and the factors may be provided once or multiple times in the course of culturing the cells.

The iPSCs of interest are mammalian, where the term mammalian refers to a cell from any animal classified as a mammal, including humans, domestic and farm animals, and zoo, laboratory, sports, or pet animals, such as dogs, horses, cats, cows, mice, rats, rabbits, etc. In some embodiments, the mammal is a human and the mammalian iPSC is therefore a human iPSC. In some cases, the iPSC was derived from a human patient.

Provided herein are PSCs (e.g., iPSCs) that include one or more CRISPR/Cas guide RNAs targeting the GATA5 promoter region (and/or one or more nucleic acids encoding the one or more CRISPR/Cas guide RNAs). In some such cases, the PSCs (e.g., iPSCs) also include a nucleotide sequence encoding a CRISPRa fusion protein (e.g., integrated into the genome or as part of an extrachromosomal nucleic acid). In some cases, the nucleotide sequence encoding the CRISPRa fusion protein is operably linked to an inducible promoter (e.g., doxycycline-regulated). Also provided herein are PSCs (e.g., iPSCs) that include an exogenous nucleic acid (e.g., integrated into the genome or as part of an extrachromosomal nucleic acid) encoding GATA5. In some such cases, the nucleotide sequence encoding GATA5 is operably linked to a promoter (e.g., constitutive promoter, inducible promoter such as a doxycycline-regulated promoter, and the like). In some cases, the PSC is a human PSC. In some cases, the iPSC is a human iPSC. In some cases, the PSC is a non-human mammalian PSC. In some cases, the iPSC is a non-human mammalian iPSC. In some cases, the PSC is a mammalian PSC. In some cases, the iPSC is mammalian iPSC.

iii. Overexpression

Subject methods include inducing GATA5 expression (i.e., overexpressing GATA5) in a pluripotent stem cell (PSC), such as in an induced pluripotent stem cell (iPSC), thereby producing a PGCLC. In some cases, GATA5 is overexpressed in a population of PSCs (e.g., iPSCs). As an example, a nucleic acid encoding GATA5 can be introduced into a population of PSCs (e.g., iPSCs) (e.g., via contacting the population with a virus to deliver the nucleic acid encoding GATA5, via contacting the population of cells with a plasmid encoding GATA5, and the like).

The term “overexpression” and “overexpressing” is used herein to refer to the act of increasing expression of a protein (i.e., increasing the amount/level of protein present) in a cell. The increase is relative to the expression/amount/level present prior to the act of overexpression. Thus, the act of stimulating/inducing expression of a protein is considered overexpressing that protein. One of ordinary skill in the art will understand how to achieve overexpression of a desired protein and any convenient method can be used.

For example, in some cases, overexpression is achieved by introducing a nucleic acid encoding the desired protein (e.g., GATA5) into the cell. In some cases, the introduced nucleic acid is DNA and will include a promoter that is operably linked to the nucleotide sequence encoding the protein (e.g., GATA5).

In some cases, the nucleic acid introduced into the cell will integrate into the genome of the cell. In some such cases, the introduced nucleic acid will include a promoter operably linked to the nucleotide sequence encoding the protein (e.g., GATA5) such that once integrated, expression of the protein (e.g., GATA5) will be under the control of the promoter of the introduced nucleic acid. In other cases, the introduced DNA will not include a promoter operably linked to the nucleotide sequence encoding the protein (e.g., GATA5)—and once integrated, expression of the protein (e.g., GATA5) will be under the control of a promoter of the cell's genome (e.g., based on where the introduced nucleic acid integrated). In some cases, the introduced nucleic acid is RNA.

In some embodiments, overexpression is achieved by stimulating/inducing expression of the desired protein (e.g., GATA5) from a promoter (e.g., constitutive, inducible) to which it is operably linked. For example, in some cases, a nucleic acid encoding the protein (e.g., GATA5) is introduced into the cell, and expression of the protein is under the control of an inducible promoter (i.e., the nucleotide sequence encoding the protein is operably linked to an inducible promoter)—and overexpression is achieved by inducing/stimulating expression from the inducible promoter (e.g., by providing the factor that induces expression). In some embodiments, expression of the desired protein (e.g., GATA5) is induced/stimulated from its endogenous promoter.

### CRISPRa Fusion Proteins

In some cases, overexpression is achieved using a CRISPRa fusion protein. Examples of such fusion proteins will be known to one of ordinary skill in the art and any convenient CRISPRa fusion protein can be used. A CRISPRa fusion protein includes a CRISPR/Cas effector protein (e.g., Cas9, Cpf1, etc.) fused to a transcription activating protein.

Examples of CRISPR/Cas effector proteins will be readily available to one of ordinary skill in the art, and any convenient CRISPR/Cas effector protein can be used. In some embodiments, a subject CRISPR/Cas effector protein will be a Cas9 protein (e.g., Staphylococcus aureus Cas9 (saCas9), Streptococcus pyogenes Cas9 (SpyCas9), Neisseria meningitidis Cas9 (nmCas9), Streptococcus thermophilus Cas9 (stCas9), etc.). In some cases, a subject CRISPR/Cas effector protein will be a Cpf1 protein.

In class 2 CRISPR systems, the functions of the effector complex (e.g., the cleavage of target DNA) are carried out by a single protein (which can be referred to as a CRISPR/Cas effector protein)—where the natural protein is an endonuclease (e.g., see Zetsche et al, Cell. 2015 Oct. 22; 163 (3): 759-71; Makarova et al, Nat Rev Microbiol. 2015 November; 13 (11): 722-36; Shmakov et al., Mol Cell. 2015 Nov. 5; 60 (3): 385-97; Shmakov et al., Nat Rev Microbiol. 2017 March; 15 (3): 169-182: “Diversity and evolution of class 2 CRISPR-Cas systems”; and Koonin et al., Curr Opin Microbiol. 2017 June: 37:67-78). As such, the term “class 2 CRISPR/Cas protein” or “CRISPR/Cas effector protein” is used herein to encompass the effector protein from class 2 CRISPR systems—for example, type II CRISPR/Cas proteins (e.g., Cas9), type V CRISPR/Cas proteins (e.g., Cpf1/Cas12a, C2c1/Cas12b, C2C3/Cas12c, Cas12d/CasY, Cas12e/CasX), and type VI CRISPR/Cas proteins (e.g., C2c2/Cas13a, C2C7/Cas13c, C2c6/Cas13b). Class 2 CRISPR/Cas effector proteins include type II, type V, and type VI CRISPR/Cas proteins, but the term is also meant to encompass any class 2 CRISPR/Cas protein suitable for binding to a corresponding guide RNA and forming a ribonucleoprotein (RNP) complex.

In some cases, the CRISPR/Cas effector protein (e.g., Cas9) is catalytically inactive (i.e., ‘dead’), which is referred to in the art as a dCas protein (e.g., dCas9). Such a protein will not exhibit the nuclease cleavage activity of the Cas effector protein, but the fusion protein (the CRISPRa fusion protein) will exhibit the activity of the protein to which the dCas protein is fused (i.e., the transcription activating protein).

A nucleic acid that binds to a CRISPR/Cas effector protein (e.g., a Cas9 protein; a type V or type VI CRISPR/Cas protein; a Cpf1 protein; etc.) (and thereby also can bind to a CRISPRa fusion protein) and targets the complex to a specific location within a target nucleic acid (e.g., target DNA) is referred to herein as a “guide RNA” or “CRISPR/Cas guide nucleic acid” or “CRISPR/Cas guide RNA.” It is understood that in some cases a guide nucleic acid includes both RNA and DNA nucleotides (i.e., is a hybrid molecule), but such a hybrid molecule is also referred to herein as a “guide nucleic acid” or “guide RNA.”

A guide RNA includes a protein-binding region to bind to the CRISPR/Cas effector protein, and also includes a targeting region that provides target specificity to the complex (the RNP complex). The targeting region includes a guide sequence (also referred to herein as a targeting sequence), which is a nucleotide sequence that is complementary to a target sequence of a target nucleic acid (e.g., target DNA).

A guide RNA can be referred to by the protein to which it corresponds. For example, when the CRISPR/Cas effector protein is a Cas9 protein, the corresponding guide RNA can be referred to as a “Cas9 guide RNA.” Likewise, as another example, when the CRISPR/Cas effector protein is a Cpf1 protein, the corresponding guide RNA can be referred to as a “Cpf1 guide RNA.”

In some embodiments, a guide RNA includes two separate nucleic acid molecules: an “activator” and a “targeter” and is referred to herein as a “dual guide RNA”, a “double-molecule guide RNA”, a “two-molecule guide RNA”, or a “dgRNA.” In some embodiments, the guide RNA is one molecule (e.g., for some CRISPR/Cas effector proteins, the corresponding guide RNA is naturally a single molecule; and in some cases, an activator and targeter are artificially covalently linked to one another, e.g., via intervening nucleotides), and the guide RNA is referred to as a “single guide RNA”, a “single-molecule guide RNA,” a “one-molecule guide RNA”, or simply “sgRNA.”

In some cases, two or more guide RNAs can be used, e.g., to target a CRISPRa fusion protein to more than one target sequence at the same time. For example, in some cases, two or more guide RNAs are used that target different sequences of a targeted promoter region (e.g., the promoter region of GATA5).

Examples of proteins (or fragments thereof) that can be used to increase transcription (i.e., transcription activating proteins) include but are not limited to: transcriptional activators such as VP16, VP64, VP48, VP64, VP160, p65 subdomain (e.g., from NFκB), Rta, VPR (which is a fusion of VP64, p65, and Rta), and activation domain of EDLL and/or TAL activation domain (e.g., for activity in plants); histone lysine methyltransferases such as SET1A, SET1B, MLL1 to 5, ASH1, SYMD2, NSD1, and the like; histone lysine demethylases such as JHDM2a/b, UTX, JMJD3, and the like; histone acetyltransferases such as GCN5, PCAF, CBP, p300, TAF1, TIP60/PLIP, MOZ/MYST3, MORF/MYST4, SRC1, ACTR, P160, CLOCK, and the like; and DNA demethylases such as Ten-Eleven Translocation (TET) dioxygenase 1 (TET1CD), TET1, DME, DML1, DML2, ROS1, and the like. See, e.g., Chavez et al., Nat Methods. 2015 April; 12 (4): 326-328. In some cases, the CRISPRa system is a SAM system, which includes 3 components that form the DNA-binding complex: (1) a CRISPRa fusion protein (e.g., dCas9 fused to VP64), (2) MS2 aptamer(s) added to the guide RNA (forming a characteristic stem loop structure recognized by MS2), and (3) transcriptional activators P65 (Nuclear Factor NF-κB p65) and HSF1 (Heat Shock Factor 1) fused with an MS2-tag corresponding to the minimal aptamer-binding peptide of the MS2 coat protein. See, e.g., review articles such as Adli, Nat Commun. 2018 May 15; 9 (1): 1911; Becirovic, Cell Mol Life Sci. 2022 Feb. 12; 79 (2): 130; and Nidhi S, et al., Int J Mol Sci. 2021 Mar. 24; 22 (7): 3327.

In some embodiments, the CRISPR/Cas effector protein of the CRISPRa fusion protein is a dCas9. In some cases, the transcription activating protein includes VPR, which as noted above is a fusion of VP64, p65, and Rta. In some cases, the CRISPRa fusion includes a dCas9 protein fused to VPR. A subject CRISPRa fusion protein can include one or more NLSs, and a nucleic acid encoding a subject CRISPRa fusion protein will in some cases be codon optimized (for a desired type of cell).

In some embodiments, a nucleic acid encoding the CRISPRa fusion protein is integrated into the genome of the PSC (e.g., iPSC). In some embodiments, the nucleic acid encoding the CRISPRa fusion protein is extrachromosomal.

In some cases, the nucleotide sequence encoding the CRISPRa fusion protein is operably linked to a constitutive promoter. In such cases, overexpression of GATA5 occurs in the presence the appropriate guide RNA(s) [targeting the GATA5 promoter]—as such, overexpression of GATA5 can be initiated by introducing the guide RNA(s). In some cases, the nucleotide sequence encoding the CRISPRa fusion protein is operably linked to an inducible promoter, and overexpression of GATA5 occurs, in the presence the appropriate guide RNA(s) [targeting the GATA5 promoter], when the appropriate factor is present to induce expression of the CRISPRa fusion protein—as such, overexpression can be initiated by introducing the appropriate factor (e.g., doxycycline) to induce expression of the CRISPRa fusion protein.

### Nucleic Acids

As would be understood to one of ordinary skill in the art, a GATA5 protein and/or a CRISPRa fusion protein can be introduced into a cell directly as protein. A GATA5 protein and/or a CRISPRa fusion protein can also be introduced as a nucleic encoding the protein (e.g., an RNA or a DNA such as an expression vector). In some cases, a GATA5 protein and/or a CRISPRa fusion protein is introduced as an RNA encoding the protein (and the cell translates the RNA into protein). In some cases, a GATA5 protein and/or a CRISPRa fusion protein is introduced as a DNA encoding the protein (and the cell expresses the protein via RNA transcription and translation into protein).

Suitable nucleic acids comprising nucleotide sequences encoding GATA5 and/or a CRISPRa fusion protein include expression vectors, where an expression vector comprising a nucleotide sequence encoding GATA5 and/or a CRISPRa fusion protein is a “recombinant expression vector.” In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to: plasmids; cosmids; minicircles; viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. Numerous suitable expression vectors are known to those of skill in the art, and many are commercially available.

Various promoters, including constitutive and inducible promoters, may be used to drive expression (e.g., of GATA5 and/or of a CRISPRa fusion protein). The promoter may be a constitutively active promoter, i.e. a promoter is active in the absence externally applied agents, or it may be an inducible promoter (e.g., T7 RNA polymerase promoter, heat shock promoter, Tetracycline-regulated promoter, Steroid-regulated promoter, doxycycline-regulated promoter, mouse metallothionein-I promoter, etc). As used herein, an inducible promoter is a promoter whose activity is regulated upon the application of an agent to the cell, (e.g. doxycycline) or the induced presence of a particular RNA polymerase (e.g., T7 RNA polymerase).

Non-limiting examples of constitutive promoters include those from cytomegalovirus (CMV) immediate early, EF1-a (also known as EF1-a), herpes simplex virus (HSV) thymidine kinase (TK), early and late SV40, long terminal repeats (LTRs) from retrovirus, and the like. Selection of the appropriate vector and promoter is well within the level of ordinary skill in the art. An expression vector may also contain a ribosome binding site for translation initiation and a transcription terminator. The expression vector may also include appropriate sequences for amplifying expression.

Methods of introducing nucleic acids (and proteins) into cells are known in the art, and any convenient method can be used. Such methods include, but are not limited to: infection (viral transduction), lipofection, electroporation, calcium phosphate precipitation, polyethyleneimine (PEI)-mediated transfection, DEAE-dextran mediated transfection, liposome-mediated transfection, nucleofection, microinjection, lipid nanoparticle formulations, and the like.

iv. Evaluation/PGCLCs

In some cases, a subject method includes a determination of whether PGCLCs were produced. In some cases, e.g., when performing the method with a population of cells, a measurement(s) (e.g., of cell markers) can be used to determine/estimate a percentage of cells or a number of cells that were successfully induced into PGCLCs. In some cases, an evaluation takes place (or the samples for evaluation, e.g., cells, RNA, and/or protein are collected) 5 or more hours after overexpression of GATA5 begins (e.g., 10 or more hours, 12 or more hours, 18 or more hours, 24 or more hours, 36 or more hours, 48 or more hours, 72 or more hours, 4 or more days, 5 or more days, 6 or more days, or 7 or more days). In some cases, an evaluation takes place (or the samples for evaluation, e.g., cells, RNA, and/or protein are collected) 5 hours to 10 days after overexpression begins (e.g., 5 hours to 8 days, 5 hours to 7.5 days, 5 hours to 7 days, 5 hours to 6 days, 5 hours to 5 days, 5 hours to 2 days, 5 hours to 3 days, 5 hours to 2 days, 5 hours to 1 day, 12 hours to 10 days, 12 hours to 8 days, 12 hours to 7.5 days, 12 hours to 7 days, 12 hours to 6 days, 12 hours to 5 days, 12 hours to 2 days, 12 hours to 3 days, 12 hours to 2 days, 12 hours to 1 day, 1-10 days, 1-8 days, 1-7.5 days, 1-7 days, 1-6 days, 1-5 days, 1-2 days, 1-3 days, 1-2 days, 2-10 days, 2-8 days, 2-7.5 days, 2-7 days, 2-6 days, 2-5 days, 2-2 days, 2-3 days, 3-10 days, 3-8 days, 3-7.5 days, 3-7 days, 3-6 days, 3-5 days, 3-2 days, 3-3 days, 4-10 days, 4-8 days, 4-7.5 days, 4-7 days, 4-6 days, 4-5 days, 4-2 days, 4-3 days, 5-10 days, 5-8 days, 5-7.5 days, 5-7 days, or 5-6 days). In some cases, an evaluation takes place (or the samples for evaluation, e.g., cells, RNA, and/or protein are collected) 1-10 days after overexpression begins (e.g., 1-8 days, 1-7.5 days, 1-7 days, 1-6 days, 1-5 days, 1-2 days, 1-3 days, 1-2 days, 2-10 days, 2-8 days, 2-7.5 days, 2-7 days, 2-6 days, 2-5 days, 2-2 days, 2-3 days, 3-10 days, 3-8 days, 3-7.5 days, 3-7 days, 3-6 days, 3-5 days, 3-2 days, 3-3 days, 4-10 days, 4-8 days, 4-7.5 days, 4-7 days, 4-6 days, 4-5 days, 4-2 days, 4-3 days, 5-10 days, 5-8 days, 5-7.5 days, 5-7 days, or 5-6 days).

### Molecular Markers

In some embodiments, one or more primordial germ cell (PGC) markers are used (after overexpression of GATA5 begins) to determine whether a PGCLC has been produced. In some embodiments, the one or more primordial germ cell markers includes at least one (e.g., at least 2, at least 3, at least 4, or at least 5) of the following markers: Eomesodermin homolog (EOMES), Transcription factor SOX-17 (SOX17), PR domain zinc finger protein 1 (PRDM1), Transcription factor AP-2 gamma (TFAP2C), Trans-acting T-cell-specific transcription factor GATA-3 (GATA3), Nanos homolog 3 (NANOS3). In other words, in some cases, the one or more (e.g., 2 or more, 3 or more, 4 or more, or 5 or more) primordial germ cell markers includes: EOMES, SOX17, PRDM1, TFAP2C, GATA3, NANOS3, or any combination thereof. In other words, in some cases, the primordial germ cell markers that are used include one or more (e.g., 2 or more, 3 or more, 4 or more, or 5 or more) markers selected from the group consisting of: EOMES, SOX17, PRDM1, TFAP2C, GATA3, NANOS3. In some embodiments, the primordial germ cell markers that are used include one or more (e.g., 2 or more, 3 or more, or 4 or more) markers selected from the group consisting of: EOMES, SOX17, PRDM1, TFAP2C, GATA3.

Because the above markers are PGC markers, expression of the above markers is indicative of PGCLCs. In some cases, expression of GATA3 is indicative of PGCLCs. In some cases, expression of SOX17 is indicative of PGCLCs. In some cases, expression of EOMES is indicative of PGCLCs. In some cases, expression of PRDM1 is indicative of PGCLCs. In some cases, expression of TFAP2C indicative of PGCLCs. In some cases, expression of NANOS3 indicative of PGCLCs. In some cases, expression of GATA3, SOX17, EOMES, and PRDM1 is indicative of PGCLCs. In some cases, expression of GATA3, SOX17, EOMES, TFAP2C, and PRDM1 is indicative of PGCLCs. In some cases, expression of PRDM1, SOX17 and EOMES is indicative of PGCLCs. In some cases, expression of PRDM1, SOX17, EOMES, and TFAP2C is indicative of PGCLCs. In some cases, expression of PRDM1, SOX17, EOMES, and NANOS3 is indicative of PGCLCs. In some cases, expression of PRDM1, SOX17, EOMES, TFAP2C, and NANOS3 is indicative of PGCLCs.

The level of expression of a biomarker can be compared to the expression level of one or more additional genes (e.g., nucleic acids and/or their encoded proteins) to derive a normalized value that represents a normalized expression level. The specific metric (or units) chosen is not crucial. In some cases, the level of expression of the marker(s) is compared to a control level, e.g., an expression level of the marker in a PSC (e.g., iPSC), or a population of such cell, in which GATA5 was not overexpressed. In some cases, the marker(s) used (see above) are considered increased by the overexpression of GATA5 if marker expression is 1.5-fold or more (e.g., 2-fold or more, 3-fold or more, 4-fold or more, 5-fold or more, 6-fold or more, or 10-fold or more) greater than a control value (e.g., value from comparable cells in which GATA5 was not overexpressed). In some cases, the marker(s) used (see above) are considered increased by the overexpression of GATA5 if marker expression is 2-fold or more (e.g., 3-fold or more, 4-fold or more, 5-fold or more, 6-fold or more, or 10-fold or more) greater than a control value (e.g., value from comparable cells in which GATA5 was not overexpressed). In some cases, the marker(s) used (see above) are considered increased by the overexpression of GATA5 if marker expression is 5-fold or more (e.g., 3-fold or more, 4-fold or more, 5-fold or more, 6-fold or more, or 10-fold or more) greater than a control value (e.g., value from comparable cells in which GATA5 was not overexpressed).

Expression of a marker can be measured using any convenient method. The terms “measuring” and “analyzing” are used herein to refer to any form of measurement, and include determining if an element is present or not. These terms include both quantitative and/or qualitative determinations. Assaying may be relative or absolute. For example, “measuring” can be determining whether the expression level is less than or “greater than or equal to” a particular threshold, (the threshold can be pre-determined or can be determined by assaying a control sample). On the other hand, “measuring to determine the expression level” or simply “measuring expression levels” can mean determining a quantitative value (using any convenient metric) that represents the level of expression (i.e., expression level, e.g., the amount of protein and/or RNA, e.g., mRNA) of a particular biomarker. The level of expression can be expressed in arbitrary units associated with a particular assay (e.g., fluorescence units, e.g., mean fluorescence intensity (MFI), threshold cycle (Ct), quantification cycle (Ca), and the like), or can be expressed as an absolute value with defined units (e.g., number of mRNA transcripts, number of protein molecules, concentration of protein, etc.).

In some cases, the RNA level is measured/detected. In some cases, the protein level is measured/detected.

### Kits

Provided are kits/systems for carrying out a subject method. Such kits comprise various combinations of components useful in any of the methods described elsewhere herein. In some embodiments a subject kit includes one or more CRISPR/CAS guide RNAs (and/or one or more nucleic acids encoding them) that target the GATA5 promoter region. In same cases, such a kit further includes a CRISPRa fusion protein (or a nucleic acid encoding same). In some cases, the nucleotide sequence encoding the CRISPRa fusion protein is operably linked to an inducible promoter (e.g., doxycycline-regulated). In some cases, a subject kit include a nucleic acid encoding GATA5 (e.g., a recombinant expression vector such as a viral vector, a plasmid, a minicircle, and the like). In some such cases, the nucleotide sequence encoding GATA5 is operably linked to a promoter (e.g., constitutive promoter, inducible promoter such as a doxycycline-regulated promoter, and the like). In some cases a subject kit includes a PSC (e.g., an iPSC).

A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods (e.g., dosing instructions, instructions to administer the component(s) to an individual. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.
1. A method of producing a primordial germ cell like cell (PGCLC), the method comprising: overexpressing GATA5 in a pluripotent stem cell (PSC), thereby producing a PGCLC.
2. The method of 1, wherein said overexpressing GATA5 comprises expressing, in the PSC, a CRISPRa fusion protein that, in the presence of a guide RNA, stimulates expression of GATA5.
3. The method of 2, wherein GATA5 expression is stimulated from its endogenous locus.
4. The method of 2 or 3, wherein a nucleotide sequence encoding the CRISPRa fusion protein is operably linked to an inducible promoter, and wherein said overexpressing GATA5 comprises stimulating expression from the inducible promoter.
5. The method of 4, wherein the nucleotide sequence encoding the CRISPRa fusion protein is integrated into the genome of the PSC.
6. The method of any one of 2-5, wherein said CRISPRa fusion protein comprises dCas9 fused to VPR.
7. The method of any one of 2-6, wherein the method comprises introducing the guide RNA or a nucleic acid encoding the guide RNA into the PSC.
8. The method of 1, wherein said overexpressing GATA5 comprises introducing a nucleic acid encoding GATA5 into the PSC.
9. The method of 8, wherein the nucleic acid encoding GATA5 is a recombinant expression vector.
10. The method of 8, wherein said overexpressing GATA5 comprises contacting the PSC with a virus comprising the nucleic acid encoding GATA5.
11. The method of 8, wherein the nucleic acid encoding GATA5 is an RNA.
12. The method of any one of 1-11, further comprising measuring expression of one or more primordial germ cell markers after overexpressing GATA5 begins to determine whether a PGCLC has been produced.
13. The method of 12, wherein said one or more primordial germ cell markers comprises: EOMES, SOX17, PRDM1, TFAP2C, GATA3, NANOS3, or any combination thereof.
14. The method of any one of 1-13, wherein the PSC is a human cell.
15. The method of any one of 1-14, wherein the PSC was derived from a human patient.
16. The method of any one of 1-15, wherein the PSC is an induced pluripotent stem cell (iPSC).

### EXPERIMENTAL EXAMPLES

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like

### Example 1: GATA5 Overexpression in iPSCs

iPSCs were derived from human skin fibroblasts from an Asian-American female with consent for commercial use (cell line generated by Takara). Briefly, human skin fibroblasts were reprogrammed into iPSCs using non-integrating vectors for the delivery of reprogramming factors Oct4, Sox2, Klf4 and c-Myc. iPSCs were maintained in mTeSR media with supplements (StemCell Technologies).

GATA5 overexpression in iPSCs was achieved using CRISPRa. Briefly, iPSCs were engineered to introduce doxycycline-inducible CRISRPa (dCas9-VPR-T2A-GFP) cassette into AAVS1 safe-harbor locus using TALEN gene editing. Subsequently, CRISPRa iPSCs were transduced with a guide RNA (gRNA) lentiviral construct expressing BFP and gRNAs targeting the GATA5 promoter. The targeted sequences were:

gRNA 1:
(SEQ ID NO: 2)
GCTGACCCTGCGGGGAAGAA
gRNA 2:
(SEQ ID NO: 3)
GTCGCCAAGCCCCGCGAGCA

Following gRNA introduction, CRISPRa expression was induced with doxycycline for 7 days. To induce GATA5 overexpression iPSCs expressing the CRISPRa machinery and gRNAs targeting GATA5 were treated with 4 uM doxycycline in mTeSR media. For both the single cell and bulk RNAseq experiments the cells were analyzed at 7 days after induction with doxycycline.

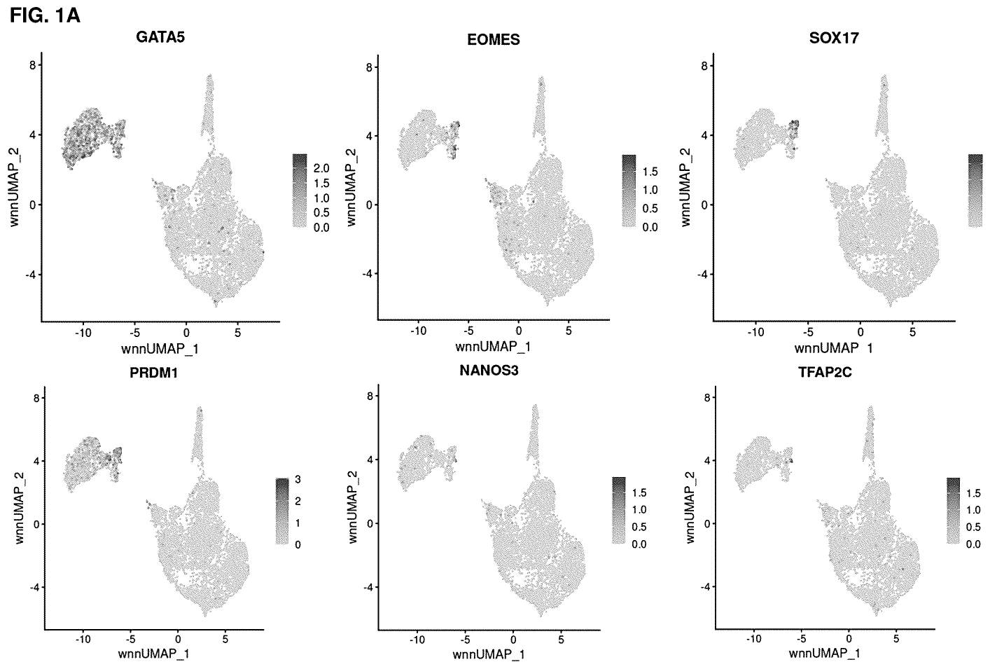

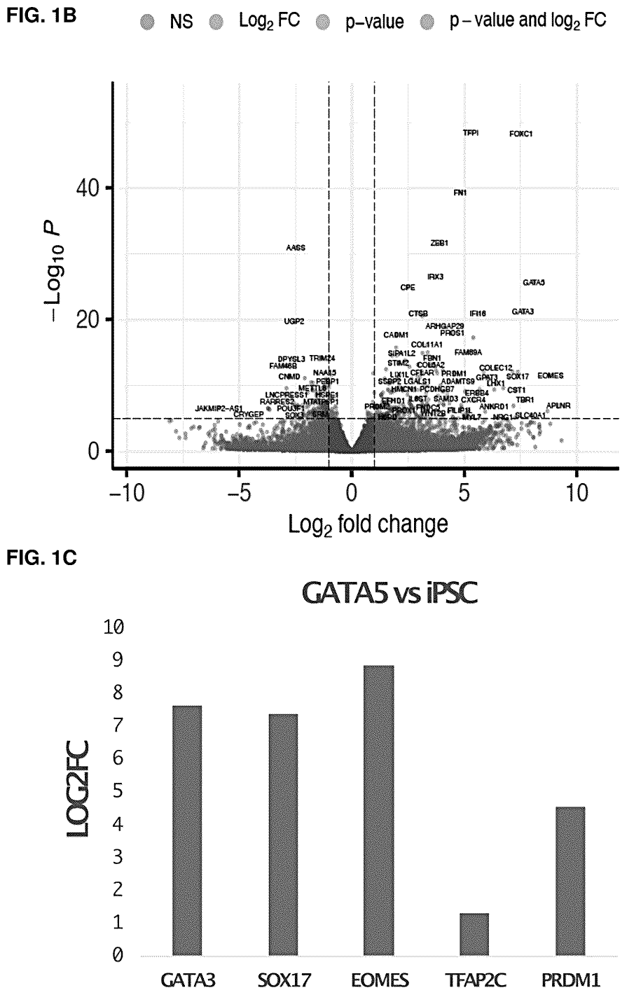

The resulting cells were then processed via 10× Genomics Chromium Single Cell Multiome ATAC+Gene Expression instructions. Profiles of the GATA5 overexpressing cells were subsequently computationally analyzed to identify differentially expressed genes and the resulting cell type (FIG. 1A). To confirm the findings, GATA5 overexpressing cells were used in a bulk RNA sequencing experiment using Lexogen QuantSeq 3′ mRNA-Seq Library Prep Kit (FIGS. 1B and 1C). Differentially upregulated genes indicate that GATA5 overexpressing cells are acquiring PGC fate.

GATA5 overexpression in iPSCs led to expression of Primordial Germ Cell markers (FIG. 1). FIG. 1A) Single cell multiomic analysis shows that GATA5 overexpressing cells express PGC markers EOMES, SOX17, PRDM1 and TFAP2C. FIG. 1B) Volcano plot from bulk RNA-seq analysis listing differentially expressed genes between GATA5 overexpressing cells and control iPSCs. FIG. 1C) Bar-graph from bulk RNA-seq experiments showing upregulation of PGC markers in GATA5 overexpressing cells and compared to iPSCs

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112 (f) or 35 U.S.C. § 112 (6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112 (6) is not invoked.

1. A method of producing a primordial germ cell like cell (PGCLC), the method comprising:
overexpressing GATA5 in a pluripotent stem cell (PSC), thereby producing a PGCLC.

2. The method of claim 1, wherein said overexpressing GATA5 comprises expressing, in the PSC, a CRISPRa fusion protein that, in the presence of a guide RNA, stimulates expression of GATA5.

3. The method of claim 2, wherein GATA5 expression is stimulated from its endogenous locus.

4. The method of claim 2, wherein a nucleotide sequence encoding the CRISPRa fusion protein is operably linked to an inducible promoter, and wherein said overexpressing GATA5 comprises stimulating expression from the inducible promoter.

5. The method of claim 4, wherein the nucleotide sequence encoding the CRISPRa fusion protein is integrated into the genome of the PSC.

6. The method of claim 2, wherein said CRISPRa fusion protein comprises dCas9 fused to VPR.

7. The method of claim 2, wherein the method comprises introducing the guide RNA or a nucleic acid encoding the guide RNA into the PSC.

8. The method of claim 1, wherein said overexpressing GATA5 comprises introducing a nucleic acid encoding GATA5 into the PSC.

9. The method of claim 8, wherein the nucleic acid encoding GATA5 is a recombinant expression vector.

10. The method of claim 8, wherein said overexpressing GATA5 comprises contacting the PSC with a virus comprising the nucleic acid encoding GATA5.

11. The method of claim 8, wherein the nucleic acid encoding GATA5 is an RNA.

12. The method of claim 1, further comprising measuring expression of one or more primordial germ cell markers after overexpressing GATA5 to determine whether a PGCLC has been produced.

13. The method of claim 12, wherein said one or more primordial germ cell markers comprises: EOMES, SOX17, PRDM1, TFAP2C, GATA3, NANOS3, or any combination thereof.

14. The method of claim 1, wherein the PSC is a human cell.

15. The method of claim 1, wherein the PSC was derived from a human patient.

16. The method of claim 1, wherein the PSC is an induced pluripotent stem cell (iPSC).


SEQUENCE LISTING (3 sequences)

SEQ ID NO: 1 - protein, 397 aa, Homo sapiens
MYQSLALAASPRQAAYADSGSFLHAPGAGSPMFVPPARVPSMLSYLSGCEPSPQPPELAARPGWAQTATADSSAFGPGSPHPPAAHPPGATAFPFAHSPSGPGSGGSAGGRDGSAYQGALLPREQFAAPLGRPVGTSYSATYPAYVSPDVAQSWTAGPFDGSVLHGLPGRRPTFVSDFLEEFPGEGRECVNCGALSTPLWRRDGTGHYLCNACGLYHKMNGVNRPLVRPQKRLSSSRRAGLCCTNCHTTNTTLWRRNSEGEPVCNACGLYMKLHGVPRPLAMKKESIQTRKRKPKTIAKARGSSGSTRNASASPSAVASTDSSAATSKAKPSLASPVCPGPSMAPQASGQEDDSLAPGHLEFKFEPEDFAFPSTAPSPQAGLRGALRQEAWCALALA

SEQ ID NO: 2 - DNA, 20 bp, Homo sapiens
  source 1..20: mol_type = genomic DNA
gctgaccctgcggggaagaa

SEQ ID NO: 3 - DNA, 20 bp, Homo sapiens
  source 1..20: mol_type = genomic DNA
gtcgccaagccccgcgagca